(19) United States
(12) Patent Application Publication
(10) Pub. No.: US 2005/0197715 A1
(43) Pub. Date: Sep. 8, 2005

(54) Methods and apparatus for implanting devices into non-sterile body lumens or organs

(75) Inventors: Chad J. Kugler, Andover, MN (US); Jerome K. Grudem JR., Rogers, MN (US); Todd A. Berg, Stillwater, MN (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Torax Medical, Inc.

(21) Appl. No.: 11/059,173

(22) Filed: Feb. 16, 2005

Related U.S. Application Data
(63) Continuation-in-part of application No. 10/134,306, filed on Apr. 26, 2002.
(63) Continuation-in-part of application No. 10/443,507, filed on May 21, 2003, now abandoned.
(63) Continuation-in-part of application No. 10/732,696, filed on Dec. 9, 2003.
(63) Continuation-in-part of application No. 10/732,693, filed on Dec. 9, 2003.
(63) Continuation-in-part of application No. 10/612,496, filed on Jul. 1, 2003.
(63) Continuation-in-part of application No. 10/802,992, filed on Mar. 16, 2004.
(60) Provisional application No. 60/547,200, filed on Feb. 23, 2004.
(60) Provisional application No. 60/547,200, filed on Feb. 23, 2004.

(51) Int. Cl. A61F 2/12

(52) U.S. Cl. 623/23.65; 623/11.11

(57) ABSTRACT

Implantable devices for use in a non-sterile environment of a patient's anatomy are medicated or include medication. For example, the housing of the device and/or members for securing the device to the patient's anatomy (e.g., the muscular esophageal wall) may be medicated to, among other things, prevent a rejection mechanism from being triggered, to prevent or reduce bacterial infection, or to promote tissue ingrowth. The medication may be for medicating tissue at the implant site, or for medicating some other portion of the patient's anatomy.

### CROSS-REFERENCES TO RELATED APPLICATIONS

This application is a continuation-in-part of U.S. patent application Ser. Nos. 10/134,306, filed Apr. 26, 2002; Ser. No. 10/443,507, filed May 21, 2003; Ser. No. 10/732,696, filed Dec. 9, 2003; Ser. No. 10/732,693, filed Dec. 9, 2003; Ser. No. 10/612,496, filed Jul. 1, 2003; and Ser. No. 10/802,992, filed Mar. 16, 2004, all of which are hereby incorporated by reference herein in their entireties.

This application also claims the benefit of U.S. provisional patent application No. 60/547,200, filed Feb. 23, 2004, which is hereby incorporated by reference herein in its entirety.

### BACKGROUND OF THE INVENTION

The present invention relates to implants that are permanently placed into a body lumen or body organ, collectively a “passageway,” of a patient. The term “passage” or “passageway” may be used herein as a generic term for tubular tissue structures or lumens and for body cavities.

In particular, the present invention relates to implants that are permanently placed in a passageway in which at least a portion of the passageway is non-sterile. In accordance with the present invention, modifications are made to the implant for such purposes as to improve infection-resistance of the implant site and/or to facilitate tissue integration to the implant relative to the passageway.

Certain passageways of the human body are non-sterile (e.g., the gastrointestinal (“GI”) tract). It is sometimes necessary to deliver an implant to these passageways. Unique design considerations may apply to delivery of an implant to a non-sterile passageway. If sterility of the implant or implant site (e.g., the esophageal wall) is compromised, germs or microorganisms, collectively “microbes,” may be introduced to the implant site in the body passageway. Implants for non-sterile passageways may therefore be susceptible to infection and the procedures for implanting the implants may leave surrounding tissue at the implant site susceptible to infection. Such infection may include a primary infection, in which the implant itself may have carried microbes into the tissues, or a secondary infection, in which the implant may have created a chronic pathway for microbes to access the tissues surrounding the implant over a period of time.

The body's immune response to such infections can cause inflammation and/or necrosis of cells and tissue which surround the implant. This reaction may affect the function of the implant and/or reduce the implant's retention to the tissue of the body passageway. Prevention of this infectious response is desirable for implant function and retention of the implant to the implant site.

Many other similar conditions can exist elsewhere in the body. For example, a sphincter in the urinary tract can become weak, resulting in urinary incontinence. The tissue surrounding or adjacent to any lumen in the body may be in need of an improvement in tone (i.e., an improvement in muscle tone or analogous to an improvement in muscle tone). Such an improvement in tone may help to reduce the size of the lumen or otherwise modify the shape or geometry of the lumen, strengthen or assist a sphincter associated with the lumen, and/or otherwise improve the performance of the lumen. In addition to the improvements in tone described above, other body lumen improvements may also be desirable. For example, such improvements may include closure or restriction of a body lumen to limit or stop the passage of gas, liquid, or solids in the body lumen (such as the urethra or bladder for incontinence control) or in a body cavity such as in the stomach or lungs.

Therefore, it would be desirable to provide improved apparatus and methods for the treatment of a dysfunctional body passage or lumen.

It is also desirable to provide improved apparatus and methods for such purposes as improving the tone of, strengthening, reinforcing, and/or reducing the size or otherwise changing the geometry of any of various lumens, organs, cavities, or similar structures in a patient's body.

### SUMMARY OF THE INVENTION

In view of the foregoing, it would be desirable to provide methods and apparatus for implanting devices into non-sterile passageways, which devices include: (1) an implant that can emit or release one or more drugs to create a microbial-free area around the implant; (2) an implant that can elute one or more drugs over a period of time to minimize infection of the implant site; (3) an implant that can have a reactive surface that can minimize infection, over a period of time, around the implant; (4) an implant, containing a material, specific geometry, and/or drug, that can facilitate the sealing off of bacteria exposure to the implant or surrounding area; and/or (5) an implant, containing a material, specific geometry, and/or drug that can facilitate the sealing off of bacteria around the implant by initiating a cell response and tissue integration to the implant.

The descriptions provided in this disclosure will be largely based on implants that are delivered into the esophagus for the treatment of gastroesophageal reflux disease (“GERD”). However, this invention will also apply to other implants delivered into the GI tract for purposes other than GERD and in locations other than the esophagus. Examples of this include stomach reduction techniques, esophageal stenting, treatment for Barrett's esophagus, etc.

One embodiment of the present invention places a porous coating on metallic fixation prongs, or constructs the prongs from a material capable of delivering a drug. These fixation prongs penetrate and remain implanted in the esophageal wall. An alternative embodiment places a porous coating on the exterior housing of an implant or constructs the housing from a material capable of delivering a drug. The fixation prongs may also be coated from a material capable of delivering a drug. Yet another embodiment places a membrane of porous material over a reservoir of medication. The term “medication” is used generically herein to include therapeutic agents of all kinds and for all purposes. Drugs are an example of medications. Ionizable metals are another example of medications. Acid neutralizers and acid blockers are still other examples of medications.

An alternative embodiment of the present invention includes administering a drug from a fluid or gel or from microspheres contained in a fluid or gel. This aspect of the invention preferably administers a fluid or gel between or within muscular layers of the stomach or esophagus.

An alternative embodiment of the present invention includes administering a drug from a suture or tension member. This aspect of the invention places a suture or tension member through or into the patient's anatomy (e.g., the esophageal or stomach wall) in order to change the shape of the anatomy or to secure an implanted feature to the targeted tissue.

Further features of the invention, its nature and various advantages will be more apparent from the accompanying drawings and the following detailed description of the preferred embodiments.

### BRIEF DESCRIPTION OF THE DRAWINGS

FIG. 1 is a simplified sectional view of a portion of a patient's internal anatomy.

FIG. 2 is a simplified sectional view of a portion of a patient's internal anatomy with the addition of an illustrative structure in accordance with the invention.

FIG. 3 is an isometric view of an implantable structure in accordance with the invention.

FIG. 4 is an isometric view of another implantable structure in accordance with the invention.

FIG. 5 is a simplified sectional view of a portion of a patient's internal anatomy with the addition of an illustrative structure and microspheres in accordance with the invention.

FIG. 6 is an implantable device shown in vivo in accordance with the invention.

FIG. 7A is an isometric view of an implantable structure with a collar for facilitating the growth of tissue in accordance with the invention.

FIG. 7B is FIG. 7A with a thread integrated in the open cells of the collar of the implantable structure in accordance with the invention.

FIG. 8A is an isometric view of an implantable structure with open cells in accordance with the invention.

FIG. 8B is a sectional view of the implantable device of FIG. 8A with threads in the open cells in accordance with the invention.

FIGS. 9A and 9B are sectional views of implantable devices with reservoirs of medication in accordance with the invention.

FIG. 10 is a simplified sectional view of a portion of a patient's internal anatomy with the addition of illustrative devices in accordance with the invention.

FIG. 11A is an isometric view of an implantable device with protrusions across the face of the device in accordance with the invention.

FIG. 11B is a simplified sectional view of a portion of a patient's internal anatomy with the addition of two of the illustrative devices shown in FIG. 11A in accordance with the invention.

FIG. 12A is a simplified sectional view of a portion of a patient's internal anatomy with the addition of a medicated tension member in accordance with the invention.

FIGS. 12B and 12C show the medicated tension member of FIG. 12A in more detail in accordance with the invention.

### DETAILED DESCRIPTION OF THE INVENTION

The present disclosure concerns, inter alia, apparatus and methods for improving the function of biological passages. The ability of biological passages to expand and contract actively or passively to regulate the flow of solids, liquids, gases, or combinations thereof, may be compromised by defects or disease. One example of a condition associated with decreased functionality of a body passage is GERD, which affects the esophagus. Other body passages that may be subject to dysfunction, defect, and disease include, but are not limited to, a fallopian tube, a urethra (for example, in the case of incontinence), and a blood vessel (for example, in the case of an aneurysm). The present invention will be described in connection with GERD and esophageal dysfunction. The invention is not limited, in all embodiments, to alleviating the symptoms of GERD and esophageal dysfunction.

The normal, healthy esophagus is a muscular tube that carries food from the mouth through the chest cavity and into the upper part of the stomach. The esophagus is composed of the esophageal wall (muscle), and the lining of the esophagus—the mucosa. The mucosa is a natural barrier to bacteria and keeps bacteria outside the sub-mucosal tissue of the esophageal wall. The esophageal wall is sterile, whereas the mucosa and the lumen of the esophagus are non-sterile.

A small-valved opening in the distal esophagus, called the lower esophageal sphincter (“LES”), regulates the passage of food into the stomach. When functioning properly, the LES presents a barrier to the reflux of acid or food back into the esophagus.

The LES also regulates the stomach intragastric pressures, regulating acidic gases from refluxing from the stomach back into the esophagus. The LES, when functioning properly, will open to vent gases from the stomach. A healthy LES at rest can resist pressure from stomach gases that are at least 10 mm Hg greater than normal intragastric pressure. This pressure difference can regulate the amount of acidic fluid that refluxes from the stomach into the esophagus.

The LES is controlled largely by two components. The primary component is intrinsic smooth muscle of the distal esophagus wall. The second component is the skeletal muscle of the crural diaphragm at the esophageal hiatus. The diaphragm is a muscle separating the stomach from the chest. The esophageal hiatus is the opening in the diaphragm where the esophagus attaches to the stomach. Studies have shown that the diaphragm may act as a sphincter around the lower end of the esophagus.

If the LES relaxes, atrophies, or degrades for any reason, the contents of the stomach, which may be acidic, are allowed back into the esophagus, resulting in reflux symptoms. The major mechanism for esophageal reflux, which may be associated with GERD, is the relaxation of one or both of the LES or hiatal diaphragm sphincter mechanisms. Normally occurring mechanisms that diminish or prevent GERD include peristaltic squeezing by the esophageal body, gravity (when a person is in an upright position), and neutralization by saliva.

Chronic or excessive acid reflux exposure may cause esophageal damage. In particular, chronic exposure to stomach gases may cause the mucosa to become inflamed or ulcerated. An inflamed or ulcerated mucosa may lead to problems that may require medical intervention. Drugs may be required to manage symptoms of the damage, and medical intervention (including surgical or endoscopic procedures) may be required to repair the damage.

Apart from an inflamed or ulcerated mucosa, other symptoms of GERD include hiatal hernias, Barrett's esophagus, dyspepsia, hemorrhage, pulmonary disorders, chronic cough, intermittent wheezing, ulcers, and esophageal cancer.

A hiatal hernia occurs when the upper potion of the stomach moves up through an opening in the diaphragm (e.g., the esophageal hiatus). If the esophageal hernia becomes enlarged (herniated), the LES function may be compromised and the risk of GERD increased.

Barrett's esophagus, a disease, occurs when the sensitive mucosa that ordinarily lines the esophagus migrates away from the lower part of the esophagus to avoid exposure to acidic fluids refluxing from the stomach. Barrett's esophagus is often a precursor to esophageal cancer.

The most common symptom of GERD is dyspepsia (commonly known as “heartburn”). Dyspepsia may be defined as an acute burning sensation in the chest area, typically behind the sternum.

One conventional surgical treatment for GERD is fundoplication. In this procedure the upper part of the stomach is wrapped around the lower part of the esophagus. This highly invasive procedure is often initially successful, but has a high risk of morbidity (including, for example, infection and bleeding).

Another conventional treatment for GERD is surgical suturing of a pleat of tissue between the LES and stomach to make the lower esophagus tighter. Suturing may be performed endoscopically using a suturing device on the end of an endoscope inserted into the esophagus through the mouth. Endoscopic procedures are less invasive than open surgery, but still require surgical incisions and great skill.

Surgery, whether endoscopic or open (such as fundoplication), may provide a basic mechanical correction. Surgical procedures may relocate and affix existing tissue of the stomach, esophagus, or both to add support and structure to the LES. LES strength is increased by the added support, thereby reducing the incidence of reflux.

Yet another conventional treatment for GERD includes the use of pharmaceutical drugs. The drugs may include acid blockers that may reduce the production of acid by the stomach. The drugs may be effective to reduce the symptoms of mild GERD, but do not treat LES dysfunction. In general, the drugs must be administered indefinitely to maintain their efficacy.

Currently, according to the American Gastroenterological Association, over $12 billion is estimated to be spent on the treatment of GERD annually in the USA alone. It is estimated that $8 billion is spent on drugs. According to a Gallup® poll, 45% of patients taking heartburn drugs report that current remedies do not relieve all symptoms and more than half agree that they would try anything new to relieve their heartburn.

In the endoscopic treatment of GERD, an implant for treatment of GERD may become non-sterile as it enters the lumen of the esophagus. If the implant is delivered into the intramuscular wall of the esophagus for the purpose of retention, the implant risks violating the natural protective barrier—the mucosa—and exposing the sterile wall. The resulting potential contamination can yield an infection response. This response may compromise the function or even the retention of the implant. Prevention of this infectious response is desirable for implant function and healthy retention in the esophagus.

In accordance with some embodiments of the present invention, acutely or chronically used devices may be implanted that have the ability to administer one or more drugs in the gastrointestinal tract or into the gastrointestinal tissues of a patient. A medication or drug would be preferably administered from the implanted device in order to affect infection, but may include and should not be limited to drug elusion to affect the healing response of the adjacent tissues. One example is a device implanted in close proximity or into the esophageal or stomach wall for the treatment of gastroesophageal reflux disease (GERD).

The potential medications or drugs that may be released from the implanted device include but are not limited to antibiotics, steroids, antiplatelets, and acid blockers. The potential antibiotics include but are not limited to Amino PCN, Aminoglycosides, Antipseudomonad PCN, Carbapenem, Cephalosporins, Fluoroquinolones, Glycopeptide, Lincomycin, Macrolide, Monobactam, Natural PCNs, Oxazolidinones, Penicillinase, Streptogramin, Sulfa, and Tetracycline.

One or more features of the implantable devices in accordance with the invention may be coated directly with a drug or include one or more biocompatible materials that have the ability to administer a drug. The drug may be administered acutely or released over an extended period of time. These materials may be biostable or bioabsorbable.

Biostable materials include but are not limited to polymers, hydrogels, porous ceramics, and porous metals. The materials are preferably permeable to the medication. Polymeric materials include but are not limited to urethanes, polycarbonates, silicones, fluoropolymers, polyesters, polypropylenes, polyethylenes, styrenes, ethylvinyl hydroxylated acetate (“EVA”), and ethylene vinyl alcohol (“EVOH”). Metallic materials include but are not limited to nickel titanium alloys, titanium, stainless steel, tantalum, platinum iridium alloys, and gold. Metallic materials may be sintered from metallic particles. The device material or coating may have differing size pores on different sections of the device or at different levels of its cross section. The drug is preferably in liquid form or a solution of medication suspended or dissolved in a solvent and is loaded onto the implant through immersion. Immersion of the implant allows the drug to absorb into the porous material. When removed from the medication and implanted into the patient, the drug is released from the pores and absorbed by the surrounding tissue structures. The release rate is related to pore size, pore density, material volume, and the molecular structure of the drug.

Bioabsorbable materials include but are not limited to poly lactic acid (“PLA”) and poly lactic-co-glycolic acid (“PLGA”). Bioabsorbable materials release an entrapped drug as the material dissolves.

Additionally, the surface of one or more features of the device may be coated with or made from a material that creates an antimicrobial area around the implant. Such a material may be silver, gold, or copper. For example, silver materials may release silver ions in a concentration sufficient to provide a localized antimicrobial effect without damaging surrounding tissues.

The metallic coating is preferably bioerodable in order to release ions. This can be achieved in several ways including but not limited to heating the implant, treating the implant with chemicals (e.g., hydrogen peroxide), or a galvanic action by adding another suitable metal to promote the release of ions.

The bioerodable property may extend the antimicrobial effect over a period of time. The rate of erosion and the amount of time for the anti-microbial property around the implant can be controlled through the coating thickness and the activation process.

A coating may be applied to the implant through known processes of vapor deposition (e.g., vacuum evaporation, sputtering, or ion plating). Alternatively, an implant may be dipped into a salt (e.g., silver chloride) solution and the solvent may be evaporated off. Salts may also be embedded in a polymeric or other suitable binder on the surface of the implant in order to have a longer lasting antimicrobial effect.

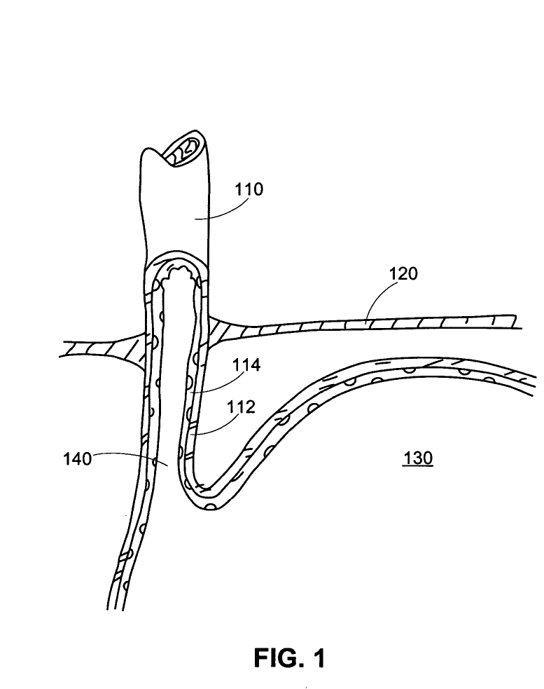

The lower part of a patient's esophagus 110 and adjacent tissue structures are shown in FIG. 1. A healthy esophagus has an outer muscular layer 112 and an internal mucosal layer 114 (i.e., the mucosa). In an unhealthy esophagus, portions of the mucosal layer may have been eroded (e.g., by GERD). The portion of the mucosa protecting the lower (closer to the stomach) portion of the esophagus is especially susceptible to erosion.

The esophagus may contain more than two distinct tissue layers but has been simplified for clarity. The implantable devices of the present invention are illustrated and described as having retention members that penetrate through the entire esophageal wall. This need not be the case and, in fact, the members of the implantable devices are preferably secured in an intramuscular layer of the esophagus. Generally speaking, the implantable devices of the present invention are preferably secured in and to soft tissue (as opposed to hard tissue such as bone or dental tissue). Additionally, the implantable devices of the present invention are preferably secured or anchored to sterile tissue and a portion of the implantable devices is preferably exposed to a non-sterile environment, all inside the body of a patient.

Other anatomical descriptions herein are also greatly simplified, both before and after treatments in accordance with this invention. Many anatomical structures and functions are in fact quite complex, to the point where they may not even be fully understood. For example, it may be convenient herein to say that the esophagus is “open” or “closed” under certain conditions (either before or after treatment in accordance with the invention), when in fact the esophagus may be only partly open when said to be “open”, only partly closed (or still partly openable without separating the magnetic devices as described below) when said to be “closed”, etc. It will therefore be understood that words like “open” and “close” and other terms and descriptions employed herein are used in a simplified (sometimes relative) sense to provide a general indication of how various anatomical structures operate, both before and after treatment in accordance with the invention.

In addition to esophagus 110, FIG. 1 shows a portion of the patient's diaphragm 120 (through which the esophagus passes), the upper part of the stomach 130, and the lower esophageal sphincter 140, which is just above the opening into the stomach and normally close to the diaphragm. The lower part of the esophagus is normally closed by sphincter 140, perhaps with some help from the adjacent diaphragm structure 120. Anything swallowed passes down esophagus 110, opening sphincter 140, and entering stomach 130. The esophagus then normally closes again. Normal pressures in stomach 130 should not cause sphincter 140 to open. But higher than normal pressures in the stomach do cause sphincter 140 to open and allow material (e.g., gas) to escape from the stomach and exit via the esophagus. In a patient with GERD, however, sphincter 140 and/or adjacent structures do not resist normal pressure in the stomach, and so material (e.g., gas or liquids) from the stomach can enter the esophagus and cause discomfort and potentially serious disease.

Preferably, when an implantable device is implanted in a patient, the patient's body does not reject the implantable device, nor does the patient's body at an implant site become infected. However, especially when implanting an implantable device in a non-sterile region of a patient's anatomy (e.g., a food passageway such as the gastro-intestinal tract) the possibility of infection may increase.

As described above, in a healthy esophagus, a protective lining—the mucosa—protects the muscular esophageal wall. The portions of the esophageal wall protected by the mucosa are sterile. The term “sterile” means that the tissue is free or substantially free from bacteria or other microorganisms. So, for example, a portion of an esophagus that is not protected by the mucosa (because it was eroded) may be non-sterile. Additionally, when an implantable device is being implanted into sterile tissue (e.g., the esophageal wall), the sterile tissue may be exposed to a non-sterile environment. To do so may result in the sterile tissue becoming bacterially infected, inflamed, or ulcerated.

When an implant is delivered into sterile tissue, there is a possibility of bacterial exposure due to delivery of the implant, but also an ongoing possibility of bacterial exposure because the implant has created one or more pathways for the bacteria to access the implant or the tissue surrounding the implant. The ability to seal the implant and surrounding area from the ongoing or future bacterial exposure may facilitate the long-term effectiveness of the implant. The combination of creating an antimicrobial area around an implant through a reactive surface or drug eluting ability, combined with the ability of the implant to create a new antimicrobial barrier to future microbial exposure in accordance with the present invention, is therefore desirable.

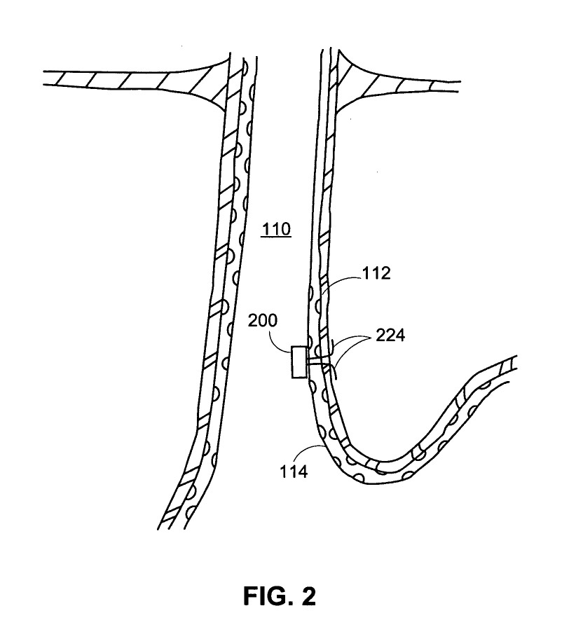

FIG. 2 shows a portion of a patient's esophagus 110 just after an implantable device 200 is secured to the esophageal wall 112. Prior to implanting implantable device 200, the entirety of the esophageal wall 112 that is shown was protected by mucosa 114 and was sterile. However, when implantable device 200 was secured to esophageal wall 112, portions of mucosa 114 are penetrated by members 224 of device 200. Members 224 are for securing device 200 to wall 112. The protective seal that mucosa 114 provides to wall 112 may be compromised, and wall 112 at members 224 may now be subject to the non-sterile environment of the esophageal lumen. Wall 112 at members 224 may therefore be subject to the possibility of bacterial infection.

Such an infection may cause erosion of mucosa 114 adjacent members 224. Additionally, insertion of members 224 into esophageal wall 112 may trigger the patient's rejection mechanism. This, generally speaking, is not desired and should be avoided if possible.

FIG. 2 is described above in the context of a portion of esophageal wall 112 protected by mucosa 114. In addition to implanting implantable devices to sterile tissue protected by a protective lining, implantable devices of the present invention may also be implanted at a non-sterile implant site. For example, an implantable device may be implanted at a portion of esophageal wall 112 not protected by mucosa 114. The muscle of esophageal wall 112 at the surface (facing inward towards the esophagus and normally protected by mucosa 114) would then not be sterile. The tissue (i.e., muscle) just below the surface is sterile. When an implantable device is implanted into sterile tissue below non-sterile tissue, the sterile tissue may be subject to the possibility of bacterial infection.

Implantable devices in accordance with the present invention are medicated to, among other things, prevent bacterial infection, foster growth of a patient's organ or lumen, including the protective lining of the organ or lumen (e.g., the mucosa), prevent the rejection of the implantable device, or treat some other malady (e.g., acid reflux).

In some embodiments, the implantable devices are not medicated to counteract any possible acute bacterial infection, but are medicated (or include medication) for treatment of some other malady. Alternatively, implantable devices of the present invention may be medicated to counteract possible bacterial infection and some other malady. An implantable device, in accordance with the invention may include two or more therapeutically different medications. These medications may be together on the device, or they may be separate on the device. For example, one medication may be disposed on the device so that it will be released at least primarily into the non-sterile region adjacent the device, while a second, therapeutically different medication may be disposed on the device so that it will be released at least primarily into the sterile region adjacent the device.

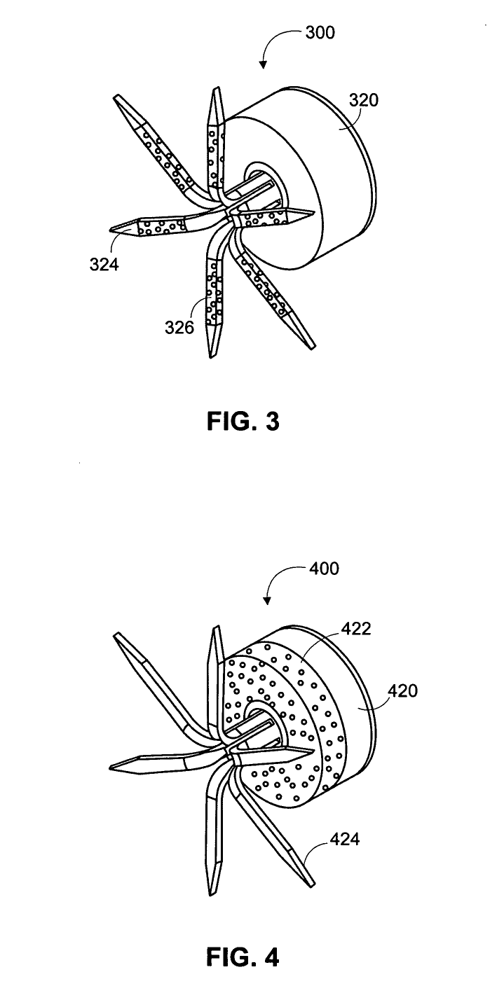

FIG. 3 shows an isometric view of an illustrative implantable device 300 in accordance with the present invention. Device 300 includes a housing or body 320 and a plurality of fixation prongs or members 324. Fixation prongs 324 are designed to be implanted into a patient's organ or lumen. As shown, prongs 324 extend radially from implant body 320 and contain segments 326. Segments 326 (1) are constructed of a reactive surface (e.g., silver, gold, or copper) capable of interfacing with surrounding tissue, (2) contain a material capable of delivering a drug, or (3) have some combination of these characteristics. Such a drug may be, for example, an acid-blocker, a steroid, an antibiotic, or a combination thereof. For enhanced effect, a segment 326 of a prong 324 may include a drug or material different than a segment 326 of a neighboring prong 324. Segments 326 may cover fixation prongs 324 in part or in their entirety.

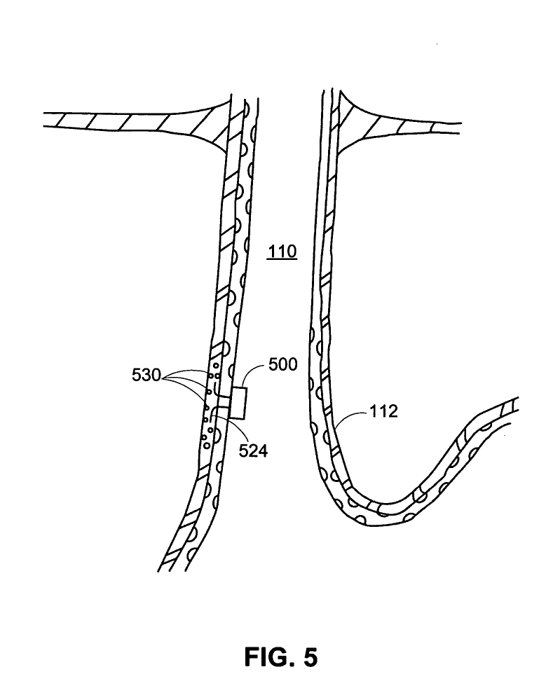

FIG. 4 shows an isometric view of another illustrative implantable device 400. Implant body 420 of device 400 is coated with one or more segments 422 of a reactive metal surface such as silver. Alternatively or in addition, segments 422 may be a porous membrane affixed to implant body 420. The porous membrane may be capable of delivering a drug. FIG. 4 shows segment 422 on a bottom portion (closer to fixation prongs 424) of implant body 420. Segment 422 could alternatively be on a top portion (away from fixation prongs 424) of implant body 420. Generally, segments 422 may cover a portion of implant body 420 or the entirety of implant body 420.

In lieu of, or in conjunction with, medicating an implantable device, bulking agents may be implanted into tissue where an implantable device is implanted or to be implanted. Bulking agents are a known technology for the purpose of being implanted or injected into the walls of the esophagus for the treatment of GERD.

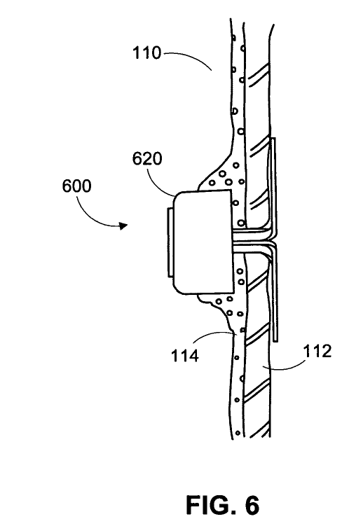

As shown in FIG. 5, implantable device 500 is implanted in a patient's esophagus 110. Bulking agents 530 are also implanted in the patient's esophagus 110—in the esophageal wall 112—where fixation prongs 524 of implantable device 500 are inserted in the wall 112.

Bulking agents 530 may be made from a polymer, a hydrogel, beads, or some other material such as a liquid. Agents 530 are inserted into the esophageal wall either by direct insertion, attachment, or injection. Agents 530 may be delivered from the non-sterile esophagus into the sterile wall. After implantation, the entry site may be exposed to contamination from bacteria. The exposure may permit infection adjacent to the implant (e.g., implantable device 500), infection of the surrounding area, and/or expulsion of the implant itself.

Bulking agents 530 may be a drug-eluting component with antimicrobial properties. For example, agents 530 may elute an acid-blocker, a steroid, an antibiotic, or a combination thereof. Additionally, the surface of agents 530 may be made of or coated with a reactive surface material such as silver to provide protection against infection.

Agents 530 may be contained in a bioerodable or biodegradable capsule that creates an antimicrobial area around the implant so that the entry site heals (e.g., the mucosa grows back) and forms a protective barrier against microbes.

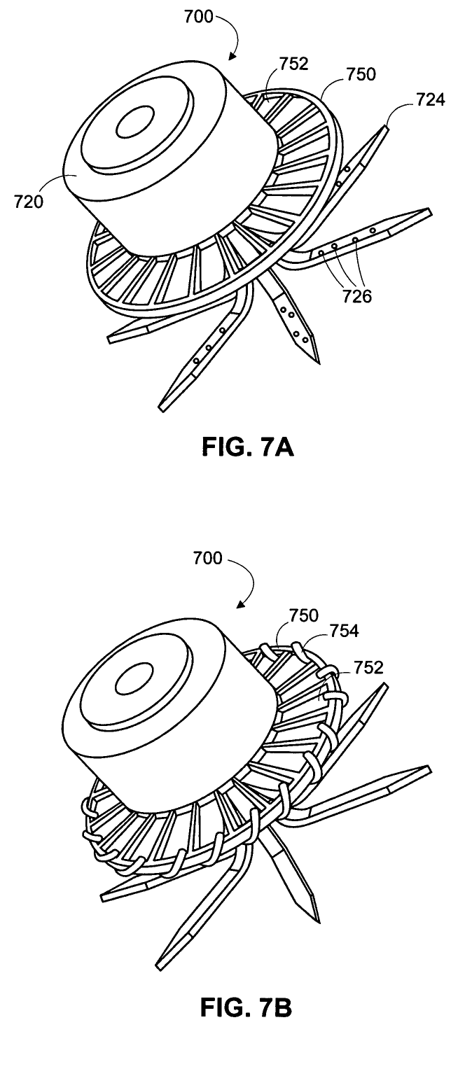

FIG. 6 shows an implantable device 600 in vivo that has been accepted (i.e., not rejected) by a patient's body. In FIG. 6, implantable device 600 was implanted into esophagus 110 of a patient. Implantable device 600 is secured to esophageal wall 112 and mucosa 114 has grown after device 600 was implanted. Mucosa 114 forms an effective antimicrobial barrier and seals esophageal wall 112 from the non-sterile environment of the inside of esophagus 110 (where food passes). As shown, mucosa 114 has advantageously grown around the exterior of body 620 of device 600.

An important adjunct to creating an antimicrobial area around an implant may be to also restore the natural barrier to microbes (e.g., the mucosa) that may have been compromised by the placement of an implant. Implantable devices may be structured such that a patient's body creates a tissue response and the growth of tissue (including a barrier to microbes) is fostered. For example, implantable devices may include threads that, in addition to the structure of the device, also foster growth of tissue.

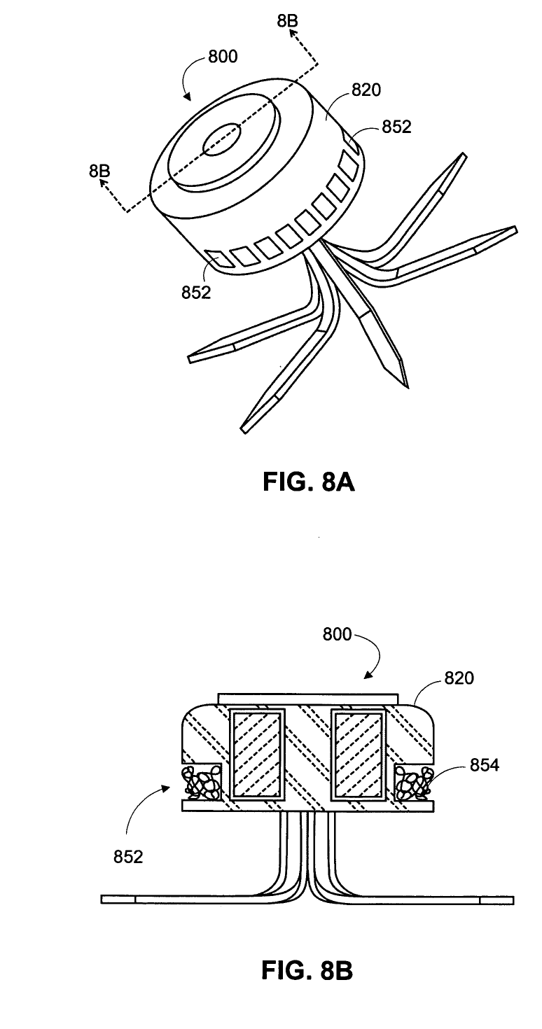

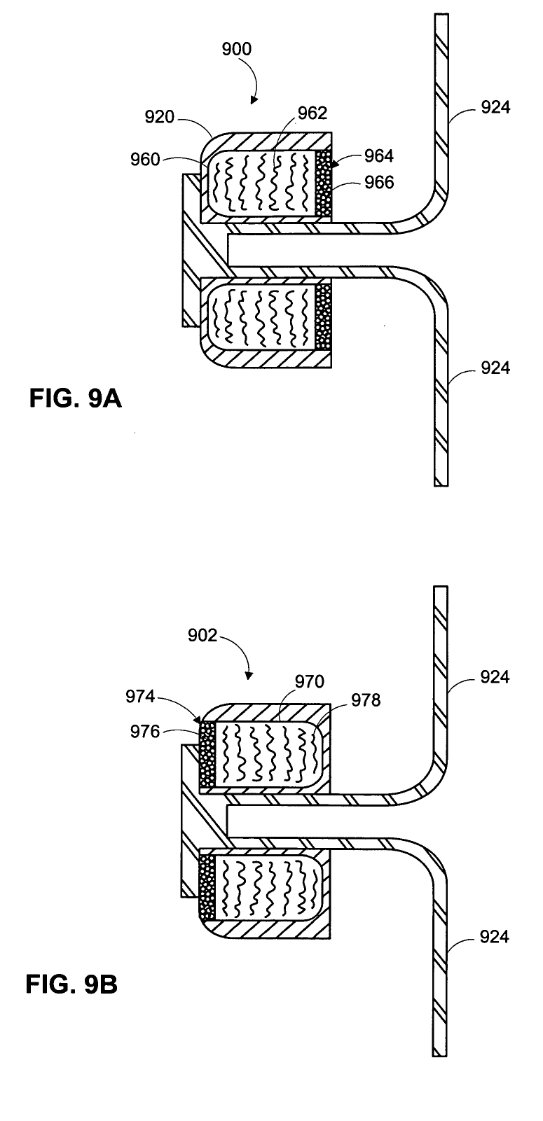

FIGS. 7A, 7B, 8A, and 8B show illustrative embodiments of implantable devices that are structured to foster growth of tissue. Implantable device 700 of FIG. 7A includes a collar 750 that is a part of or affixed to implant body 720 of implantable device 700. Collar 750 is designed to facilitate tissue ingrowth around the open cells 752 in collar 750. Additionally, retention fingers 724 may contain holes 726 to anchor new cell growth.

FIG. 7B shows thread 754 integrated into the collar 750 of device 700. For example, thread 754 may be threaded through open cells 752 of collar 750. Thread 754 may be made of, for example, polyester, any other polymer, or metal. Thread 754 may be a single thread or multiple threads (a fiber), may be woven or non-woven, and may also be medicated. Thread 754 may provide a more desirable or aggressive tissue response due to a cell reaction to the material. Thread 754 may also create more surface area to which the tissue may react and attach.

FIGS. 8A and 8B show implantable device 800 with an implant body 820 modified to create a mechanical matrix of open cells 852, which can act as cavities for tissue ingrowth and sealing. FIG. 8B is a section view of implantable device 800 of FIG. 8A through line 8B-8B. Cells 852 may also be filled with thread 854 (FIG. 8B) to further facilitate a tissue response and sealing of the implantable device 800. Thread 854 may be a single thread or multiple threads, may be polyester, any other polymer, or metal. Additionally, thread 854 may be woven or non-woven.

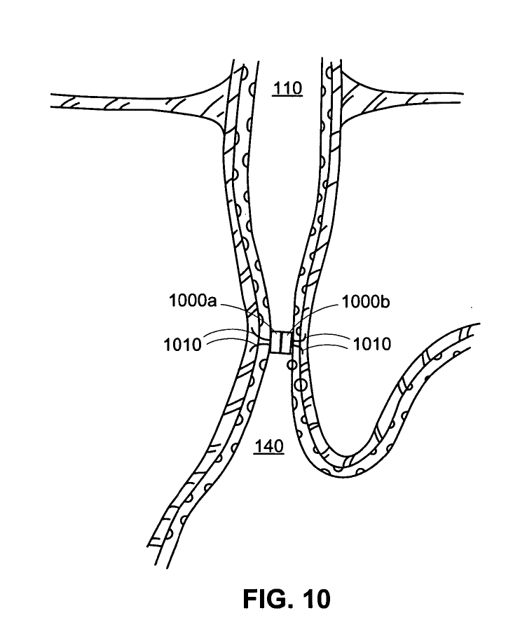

Turning to FIG. 9A, implantable device 900 includes a reservoir 960 of medication 962. In the embodiment shown in FIG. 9A, reservoir 960 is internal to body 920 of device 900. Reservoir 960 has an opening 964 at the bottom of body 920 (that portion of body 920 facing members 924).

Opening 964 may be covered by a porous membrane 966 for controlled release of the medication 962 included in reservoir 960. Pores in porous membrane 966 may be sized specifically for how fast (or slowly) medication 962 is to be released from reservoir 960.

Implantable device 902 of FIG. 9B is similar to implantable device 900 except that reservoir 970 of device 902 has an opening 974 facing towards the top of body 980 (facing away from members 924). Opening 974 may be covered by a porous membrane 976 for controlled release of the medication 978 included in reservoir 970.

Medication 962 and 978 can be for healing tissue at the implant site or for treating some other malady. If, for example, implantable device 902 is implanted in the esophagus, medication 978 may be an acid-blocker for reducing the amount of acid produced by the stomach.

Implantable devices 900 and 902 are described above as having one reservoir each. Devices in accordance with the present invention are not limited to having one reservoir. When implantable devices have more than one reservoir, each of the reservoirs may have a separate opening facing in a different direction. For example, an implantable device may have two reservoirs. In such embodiments, medication stored in a first reservoir may be for promoting the healing of tissue at an implant site. Medication stored in a second reservoir may be for treatment of a malady away from the implant site. So, for example, a first reservoir with an opening facing towards tissue at the implant site may be for combating bacterial infection and promoting regrowth of tissue (e.g., the mucosa). The second reservoir, with an opening facing away from the implant site, may be for some other treatment (e.g., reducing the amount of acid produced by the stomach).

In some embodiments of the present invention, a pair or more of implantable devices including magnets or ferromagnetic material are delivered intra-murally into the esophagus. A delivery catheter is inserted trans-orally and advanced to the site of the LES. A first implantable device including a magnet is then advanced from the delivery catheter into the wall of the esophagus. A second implantable device with a matching (i.e., opposite polarity) magnet is delivered separately. The two devices add bulk to the LES and add tone or pressure to the LES. Under transient relaxation of the LES, the sphincter may open at low pressures, allowing reflux. To overcome this low pressure relaxation, the magnetic force of the magnets is added to the closing tone pressure of the LES. The amount of this magnetic force can be tailored to an individual's clinical requirement. Since the LES is mostly closed, the opposing magnets will prevent migration. The delivery of these systems can be aided by direct visual, x-ray, and/or echo. The echo will be used to determine the depth at which the magnets are delivered into the wall of the esophagus.

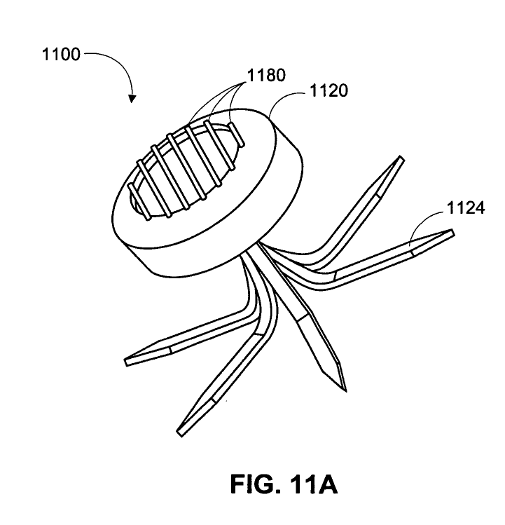

FIG. 10 shows the end result of treatment of a patient for GERD in accordance with an illustrative embodiment of the invention. In FIG. 10, two implantable devices 1000a and 1000b have been implanted in the patient's esophagus in the vicinity of esophageal sphincter 140. Implantable devices 1000a and 1000b are magnetic or include a magnet. At least a portion of each of devices 1000a and 1000b is medicated or devices 1000a and 1000b include a housing that stores medication.

In the embodiment shown in FIG. 10, at least one of devices 1000 is actively magnetic. An actively magnetic device (e.g., a permanent magnet or an electromagnet) is a source of a magnetic field. The other of devices 1000 may be either actively magnetic or passively magnetic (e.g., a body of initially unmagnetized ferro-magnetic material). Thus, phrases like “magnetic device,” “magnetic material,” etc. generally refer to both actively and passively magnetic devices, materials, etc. However, it will be understood that in any system of multiple magnetic devices there should be at least one actively magnetic device.

In the particular embodiment shown in FIG. 10, magnetic devices 1000a and 1000b are implanted in esophagus 110 so that they magnetically attract one another and help to hold the esophagus closed in their vicinity. There are many ways that implantable devices in accordance with there present invention such as devices 1000 can be implanted, as described in U.S. patent application Ser. No. 10/612,496, filed Jul. 1, 2003.

In the illustrative embodiment shown in FIG. 10, each of devices 1000a and 1000b is implanted on a respective diametrically opposite side of the esophageal lumen. The preferred axial location for devices 1000 along the longitudinal axis of the esophagus is adjacent lower esophageal sphincter 140. Devices 1000 are implanted on or in the inner wall surface of the esophageal lumen in this embodiment. A possible advantage of this type of surface-implanting is that there is then no tissue between magnetic devices 1000a and 1000b when those devices are able to be pulled together (as shown in FIG. 10) by the magnetic attraction between them. This tends to give better fore-knowledge of the end-point magnetic attraction between devices 1000. Magnetic attraction drops off rapidly as the distance between devices 1000 increases. Tissue thicknesses can vary. If one or more tissue thicknesses are between devices 1000 when they are closest together, it can be more difficult to predict how strong the end-point magnetic attraction will be. But if there is no tissue between devices 1000 when they are closest together in the patient, the magnitude of the end-point magnetic attraction should be the same as when the devices are outside the patient prior to being implanted. In other words, the in vivo end-point magnetic attraction can be more easily designed into the devices if no variable tissue thickness comes between those devices when they are closest together in vivo.

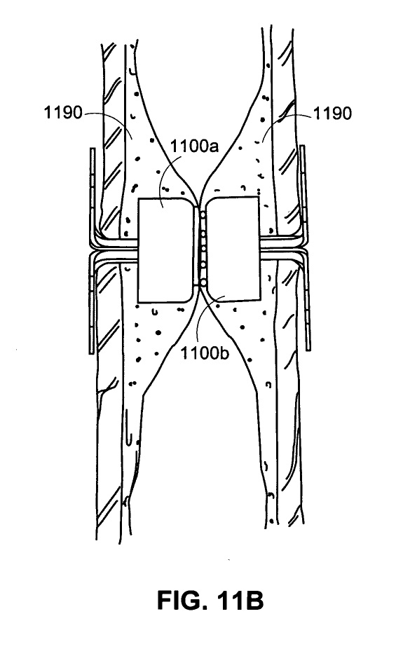

FIG. 11A shows an implantable device 1100 with a modified body 1120. Implantable device 1100 is modified such that there is no tissue thickness between implantable devices 1100 when they come together in vivo. In the embodiment described in connection with FIGS. 11A and 11B, implantable devices 1100 include ferromagnetic material. The face of body 1120 (the portion of body 1120 furthest away from members 1124 of device 1100) has bar-like protrusions 1180. When respective implantable devices 1100 are implanted in an organ or lumen to alter the anatomy of that organ or lumen (e.g., altering the shape of the LES), protrusions 1180 of respective implantable devices 110 are preferably matched (e.g., in a first implant the protrusions are off-set 90 degrees from the protrusions of the second implant) (see, for example, FIG. 11B).

Protrusions 1180 allow cell growth across the face of implantable device 1100 without limiting the ability of two implantable devices 1100 of opposite magnetic polarity to close. When two implantable devices of this type close, only protrusions 1180 of the respective implantable devices 1100 come in contact. Protrusions 1180 allow the cell growth to cover the faces of the implant while still allowing the magnetic attraction between the two devices to pull them together. In FIG. 11B, tissue ingrowth 1190 covers both implants 1100a and 1100b, including the faces of the implants (but not protrusions 1180). In other words, a surface of each implant 1100 is textured (1180) so that tissue can grow over a portion of that surface while leaving other portions of that surface exposed for contact with the other implant that faces toward the first-mentioned implant.

Reverting back to FIG. 10, the strength of the magnetic attraction between devices 1000a and 10b can be any amount that is helpful to keep esophagus 110 at least partly closed in the absence of material (e.g., food or liquid) moving down the esophagus or in the absence of higher than normal stomach pressure that should produce some escape of material (e.g., gas) up the esophagus. For example, the end-point magnetic attraction between devices 1000a and 1000b may be in the range from about log to about 500 g of force. The amount of force thus employed may depend on the clinical application of the technology, various clinical applications being mentioned throughout this specification.

Many different securing techniques can be used for magnetic devices 1000a and 1000b. It will suffice to note that FIG. 10 shows that each of devices 1000a and 1000b has two sharply pointed prongs 1010 that extend out from the rear of the associated device in directions that diverge from one another away from the remaining, main body of the associated device. In the illustrative embodiment shown in FIG. 10 each of prongs 1010 is made of metal, preferably a highly elastic, resilient metal such as nitinol. In this embodiment prongs 1010 are resiliently biased to assume the positions shown in FIG. 10. The prongs 1010 of each device 1000 preferably penetrate the tissue of the esophagus, perhaps first entering that tissue relatively parallel to one another, and then spreading apart in or beyond the tissue to secure the device to the tissue and resist removal of the device from the tissue. The free ends of prongs 1010 are preferably sharpened to facilitate this penetration of the tissue by the prongs. FIG. 10 shows the tissue as essentially a two-layer structure. But the tissue structure may in fact have even more layers than this, depending to some extent on how closely one analyzes the structure. Prongs 1010 may penetrate this tissue structure to any desired degree. In the embodiment shown in FIG. 10 prongs 1010 are shown passing through a superficial inner layer of the tissue structure and entering a more muscular (and therefore stronger) outer layer of the tissue. It is desirable for the attachment structure to engage some relatively strong tissue structure to ensure good retention of devices 1000. An alternative to what is shown in FIG. 10 is to have the retention structure such as prongs 1010 pass almost all the way through the associated tissue structure.

Implantable devices 300, 400, 500, 600, 700, 800, 900, 902, and other implantable devices in accordance with the present invention may also contain ferromagnetic material. The ferromagnetic material may be a magnet and the material may be actively magnetic or passively magnetic.

FIG. 10 illustrates an embodiment using devices of the present invention in which the shape of a patient's organ or lumen is altered. In FIG. 10, the devices used to alter the shape of a patient's organ or lumen, devices 1000a and 1000b, both include ferromagnetic material. Implantable devices of the present invention need not include ferromagnetic material to alter the shape of a patient's organ or lumen. For example, tension members may be implanted in, around, or through one or more organs or lumens of a patient to alter the shape of one or more organs or lumens.

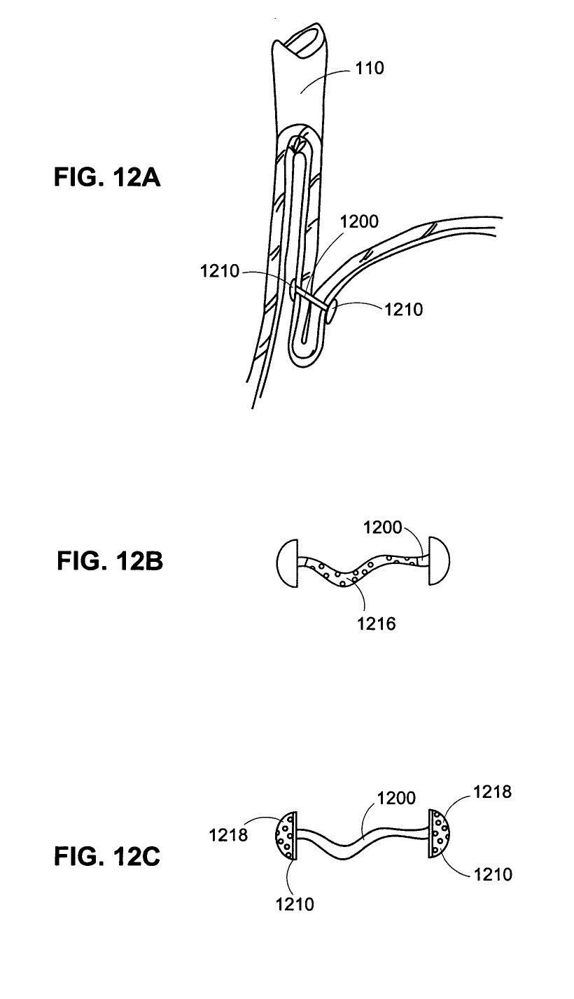

FIG. 12A shows a section view of the distal esophagus 110 with tension member 1200 placed through or into the esophageal wall. FIG. 12A shows a pledget 1210 at each end of the tension member. This structure retains the tissue or provides sufficient volume to reduce the esophageal cross section. The tension member can be a continuous loop, where the ends are secured by tying a knot or other securement means. In this case, the pledgets can be eliminated. FIG. 12B shows tension member 1200 with section 1216 capable of delivering a drug or having a reactive surface such as silver. FIG. 12C shows tension member 1200 with pledgets 1210 and sections 1218 containing a material capable of delivering a drug or having a reactive surface such as silver.

Although the present invention has been illustratively discussed primarily in the context of treating GERD, the invention has many other possible applications, as will be readily apparent to those skilled in the art from this specification. Examples of its various possible applications include treatment of a wide variety of body passages, organs, or cavities in the digestive, respiratory, circulatory, reproductive, and excretory systems.

1. An implant structure for disposition inside a patient's body comprising:
a first portion adapted to be resident, in use, in a non-sterile region inside the body;
a second portion adapted to pass, in use, from the non-sterile region into a sterile region of the body to secure the implant structure in place; and
medication.

2. The implant structure defined in claim 1 wherein the medication is adapted for release, in use, from the implant structure into the patient.

3. The implant structure defined in claim 2 wherein the medication is at least partly disposed for release, in use, into the non-sterile region.

4. The implant structure defined in claim 2 wherein the medication is at least partly disposed for release, in use, into the sterile region.

5. The implant structure defined in claim 2 wherein the medication comprises:
therapeutically different first and second medications.

6. The implant structure defined in claim 5 wherein the first and second medications are physically separate from one another.

7. The implant structure defined in claim 5 wherein the first medication is adapted for treatment of the non-sterile region, and wherein the second medication is adapted for treatment of the sterile region.

8. The implant structure defined in claim 2 wherein the medication is further adapted to help maintain, in use, sterility of the sterile region.

9. The implant structure defined in claim 2 wherein the medication is further adapted to combat possible infection, in use, along the implant structure from the non-sterile region into the sterile region.

10. The implant structure defined in claim 1 wherein the first portion is adapted to be resident, in use, in the lumen of the patient's esophagus, and wherein the second portion is adapted to, in use, secure the implant structure to the patient's esophagus.

11. The implant structure defined in claim 1 wherein the medication is at least partly selected from the group consisting of an acid blocker, a steroid, an antibiotic, and combinations thereof.

12. The implant structure defined in claim 1 wherein the medication includes a metal that is ionizable in use.

13. The implant structure defined in claim 12 wherein the metal is at least partly selected from the group consisting of silver, gold, copper, and combinations thereof.

14. A method of treating a patient comprising:
delivering an implant structure into the patient so that a first portion of the implant structure is disposed in a non-sterile region inside the patient;
disposing a second portion of the implant in a sterile region inside the patient to secure the implant structure in place in the patient; and
releasing medication from the implant structure into the patient.

15. The method defined in claim 14 wherein the delivering is performed via a non-sterile lumen of the patient.

16. The method defined in claim 14 wherein the disposing comprises:
passing the second portion from the non-sterile region into the sterile region.

17. The method defined in claim 14 wherein the releasing comprises:
releasing at least some of the medication into the non-sterile region.

18. The method defined in claim 14 wherein the releasing comprises:
releasing at least some of the medication into the sterile region.

19. The method defined in claim 14 wherein the medication includes first and second medications that are therapeutically different from one another, and wherein the releasing comprises:
releasing the first and second medications into respective different locations in the patient.

20. The method defined in claim 19 wherein the releasing the first and second medication comprises:
releasing the first medication at least primarily into the non-sterile region; and
releasing the second medication at least primarily into the sterile region.

21. The method defined in claim 14 further comprising:
implanting a bulking structure in the sterile region adjacent the second portion.

22. The method defined in claim 21 wherein the bulking structure includes further medication and wherein the method further comprises:
releasing the further medication from the bulking structure into the sterile region.

23. An implant structure for disposition inside a patient's body comprising:
a first portion adapted to be resident, in use, in a non-sterile region inside the body;
a second portion adapted to be resident, in use, in a sterile region inside the body to secure the implant structure in place; and
medication that is adapted for release, in use, from the implant structure into the patient.

24. The implant structure defined in claim 23 wherein the second portion is adapted to be resident, in use, in soft body tissue of the patient.

25. The implant structure defined in claim 23 wherein at least some of the medication is disposed for release, in use, into the sterile region.

26. The implant structure defined in claim 23 wherein at least some of the medication is disposed for release, in use, into the non-sterile region.

27. The implant structure defined in claim 23 wherein the medication includes therapeutically different first and second medications.

28. The implant structure defined in claim 27 wherein the first medication is disposed for release, in use, at least primarily into the sterile region.

29. The implant structure defined in claim 28 wherein the second medication is disposed, for release, at least primarily into the non-sterile region.

30. The implant structure defined in claim 23 wherein at least one of the first and second portions is adapted to promote, in use, tissue growth to the implant structure.

31. The implant structure defined in claim 30 wherein the at least one portion includes open cells adapted to promote, in use, tissue growth to the implant structure.

32. The implant structure defined in claim 30 wherein the at least one portion includes at least one thread adapted to promote, in use, tissue growth to the implant structure.

33. The implant structure defined in claim 23 wherein the first portion includes a collar structure adapted to seal, in use, around any penetration into the sterile region by the second portion.

34. The implant structure defined in claim 23 wherein at least one of the first and second portions includes magnetic material.

35. The implant structure defined in claim 34 wherein a surface of the first portion that is remote from the second portion is textured so that, in use, tissue can grow over parts of that surface while leaving other parts of that surface exposed for contact with another implant structure that, in use, faces toward that surface.

36. The implant structure defined in claim 23 wherein at least some of the medication is disposed in a reservoir in at least one of the first and second portions.

37. The implant structure defined in claim 23 further comprising a porous structure covering the reservoir.

38. The implant structure defined in claim 37 wherein the porous structure is adapted to, in use, influence a rate of release of medication from the reservoir.

39. The implant structure defined in claim 23 wherein at least some of the medication is disposed in a coating on at least a part of at least one of the first and second portions.
